(19) United States
(12) Patent Application Publication
(10) Pub. No.: US 2013/0045907 A1
(43) Pub. Date: Feb. 21, 2013

(54) Methods of Reducing Odor

(71) Applicants: Anthony Charles Lanzalaco, Fairfield, OH (US); Cynthia Marie Stark, Fairfield, OH (US); Andrea Kay Heape, Montgomery, OH (US)

(72) Inventors: Anthony Charles Lanzalaco, Fairfield, OH (US); Cynthia Marie Stark, Fairfield, OH (US); Andrea Kay Heape, Montgomery, OH (US)

(21) Appl. No.: 13/585,921

(22) Filed: Aug. 15, 2012

Related U.S. Application Data
(60) Provisional application No. 61/523,738, filed on Aug. 15, 2011.

(51) Int. Cl. A61K 8/58; A61Q 15/00; A61Q 19/10; A61K 8/02; A61K 8/27

(52) U.S. Cl. 510/133; 510/131

(57) ABSTRACT

Methods for reducing odor include applying and/or depositing particulate antimicrobial agents via a rinse-off personal care composition to the skin and/or hair follicles.

### CROSS REFERENCE TO RELATED APPLICATION

This application claims priority to U.S. Provisional Application Ser. No. 61/523,738 filed on Aug. 15, 2011, which is incorporated herein by reference.

### TECHNICAL FIELD

The present disclosure generally relates to methods of reducing odor by depositing and/or applying at least a portion of a particulate antimicrobial agent into a hair follicle via a rinse-off personal care composition.

### BACKGROUND

It is well established that human underarm malodors are caused by microbial interactions with apocrine gland secretions. Historically, people have attempted to reduce these odors through cleansing and the topical application of deodorant or antiperspirant products. However, it has been observed that even the combination of potent antimicrobials, strong masking perfumes, and rigorous underarm cleansing may not be sufficient to eliminate malodor. Accordingly, it is desirable to provide improved methods for reducing malodor.

### SUMMARY

A method of reducing odor comprises depositing at least a portion of a particulate antimicrobial agent into a hair follicle, wherein the particulate antimicrobial agent has an average particle size less than about 50 μm and the particulate antimicrobial agent is applied to the hair follicle via a rinse-off personal care composition.

A method of reducing odor comprises applying a rinse-off personal care composition to at least a portion of hair follicles on skin, wherein the rinse-off personal cleansing composition comprises from about 0.025% to about 1% particulate zinc pyrithione and from about 0.2% to about 2% of zinc carbonate and wherein the zinc pyrithione has an average particle size of less than about 10 um and zinc carbonate has an average particle size less than about 50 μm.

### BRIEF DESCRIPTION OF THE DRAWINGS

FIG. 1 is a chart showing the antibacterial results for a control composition versus a ZPT/ZnCO₃ composition;

FIG. 2 is a chart showing the 12 hour odor score for a control composition versus a ZPT/ZnCO₃ composition;

FIG. 3 is a chart showing the 24 hour odor score for the control composition versus the ZPT/ZnCO₃ composition;

FIG. 4 is a chart showing the 12 hour odor score for a control composition versus a second ZPT/ZnCO₃ composition; and

FIG. 5 is a chart showing the 24 hour odor score for the control composition versus the ZPT/ZnCO₃ composition.

### DETAILED DESCRIPTION

### I. Definitions

As used herein, the following terms shall have the meaning specified thereafter:

“Anhydrous” refers to those compositions, and components thereof, which are substantially free of water.

“Bar soap” refers to compositions intended for topical application to a surface such as skin or hair to remove, for example, dirt, oil, and the like. The bar soaps can be rinse-off formulations, in which the product is applied topically to the skin or hair and then subsequently rinsed within minutes from the skin or hair with water. The product could also be wiped off using a substrate. Bar soaps can be in the form of a solid (e.g., non-flowing) bar soap intended for topical application to skin. The bar soap can also be in the form of a soft solid which is compliant to the body. The bar soap additionally can be wrapped in a substrate which remains on the bar during use.

“Leave-on composition” refers to a composition that is placed on its intended target, like the skin, and is left in place for an extended period of time, generally hours, in order to provide its benefit, like a stick antiperspirant.

“Personal care composition” refers to compositions intended for topical application to skin or hair. The personal care compositions can be, for example, in the form of a liquid, semi-liquid cream, lotion, gel, or solid and are intended for topical application to the skin and/or hair. Examples of personal care compositions can include but are not limited to bar soaps, shampoos, conditioning shampoos, body washes, moisturizing body washes, shower gels, skin cleansers, cleansing milks, in shower body moisturizers, pet shampoos, shaving preparations, etc.

“Rinse-off” means the intended product usage includes application to skin and/or hair followed by rinsing and/or wiping the product from the skin and/or hair within a few seconds to minutes of the application step.

“STnS” refers to sodium trideceth(n) sulfate, wherein n can define the average number of moles of ethoxylate per molecule.

“Structured” refers to having a rheology that can confer stability on the personal care composition. A cleansing phase can be considered to be structured if the cleansing phase has one or more following characteristics: (a) Zero Shear Viscosity of at least 100 Pascal-seconds (Pa-s), at least about 200 Pa-s, at least about 500 Pa-s, at least about 1,000 Pa-s, at least about 1,500 Pa-s, or at least about 2,000 Pa-s; (b) A Structured Domain Volume Ratio as measured by the Ultracentrifugation Method described hereinafter, of greater than about 40%, at least about 45%, at least about 50%, at least about 55%, at least about 60%, at least about 65%, at least about 70%, at least about 75%, at least about 80%, at least about 85%, or at least about 90%; or (c) A Young's Modulus of greater than about 2 Pascals (Pa), greater than about 10 Pa, greater than about 20 Pa, greater than about 30 Pa, greater than about 40 Pa, greater than about 50 Pa, greater than about 75 Pa, or greater than about 100 Pa.

“Substantially free of” refers to about 2% or less, about 1% or less, or about 0.1% or less of a stated ingredient. “Free of” refers to no detectable amount of the stated ingredient or thing.

### II. Reduction of Malodor

Researchers are puzzled by the less than expected effectiveness cleansing and topical application of antimicrobials have had on bacteria generating underarm odors. One explanation as to why conventional antimicrobial intervention has not been as successful at reducing underarm odors as theoretically predicted is that the historical underarm malodor model was incomplete.

In our efforts to more completely model underarm malodor formation and control, we have discovered that a significant factor limiting the effectiveness of conventional therapies is their relative inability to access malodor causing bacteria. We have observed that malodor causing bacteria are often present within the hair follicles and skin crevices and those conventional cleansing products do a poor job of reaching and controlling bacteria in these areas. This is especially true in the high malodor area of the underarm where a unique combination of factors is present. The distinguishing feature of the underarm is the presence of three secreting glands—eccrine, sebaceous and apocrine—in an area densely populated with hair follicles and skin crevices.

Hair follicles and skin crevices offer bacteria an ecologically protected area that is relatively inaccessible during conventional cleaning. The underarm secreting glands provide the bacteria with water, nutrients, and odor precursor materials. In this environment, bacteria are free to metabolize odor precursor compounds producing malodors that can overwhelm even the strongest masking perfumes.

Current rinse-off hygiene formulations, particularly body washes and bar soaps are not designed to target odor producing bacteria in the hair follicle and skin crevices. During typical use, these formulations are applied to the underarm and then rinsed away only targeting bacteria on the surface of the skin. This results in only a transient reduction in underarm odor as the antibacterial agents are washed away during rinsing. Leave-on commercial products that are specifically targeted for underarm applications, such as various forms of antiperspirant and deodorant formulations, are typically left on the underarm after application and do not suffer from the limitations associated with rinse off cleansing products i.e. the antibacterial agent or masking agent are not rinsed from the skin. Therefore, the discovery of the unique role that hair follicles and skin crevices play in shielding malodor producing bacteria from daily hygiene measures has enabled the identification of a novel approach for reducing underarm malodor.

Surprisingly, a substantial anti-odor benefit was found when particulate antimicrobial agents described herein were deposited into skin crevices and/or hair follicles. Even more surprisingly, this anti-odor benefit was seen in rinse-off personal care compositions.

Rinse-off personal care compositions described herein can be applied as conventional rinse-off formulations, but unlike conventional rinse-off formulations these rinse-off personal care compositions leave behind antimicrobial agents designed to survive the water rinse off enabling them to reduce malodor associated with various parts of an individual's body. For example, an individual applying a body wash to a targeted area (e.g., underarm) via their hands or other applicator (e.g., sponge) can clean the targeted area with the body wash and then wash with water as they typically would, once a day. However, by using a body wash having the rinse-off personal care composition as described herein, the particulate antimicrobial agents are effectively deposited within skin crevices and/or hair follicles upon application of the personal care composition, such that during the wash/rinse period these antimicrobial agents are not entirely washed away. Even when the targeted area is dried the antimicrobial agents remain substantially deposited within the skin crevices and/or hair particles. With such targeted deposition of the antimicrobial agents the amount of malodor formed by bacteria is substantially decreased and odors take substantially longer to form. In fact, by applying such rinse-off personal care compositions as described, an individual can minimize the use of antiperspirants and deodorants if desired.

For example, Table 1, below, shows the Hair Pluck (antibacterial) results for two clinical trials that assessed the effect of body wash compositions having various levels of particulate antimicrobial agents (Trials 1 and 2). Trial la represents a control having no particulate antimicrobial agent within the body wash formulation. Trial 1a shows very little change in the baseline detection time indicating little to no follicular antibacterial benefit from the control.

Trial 1b shows that the addition of 1.6% by weight ZnCO₃, alone, to the control rinse off chassis did not enhance the follicular antibacterial efficacy above what was observed for the control formulation. This indicates little or no follicular antibacterial activity for this composition. It should be noted that the delivered underarm dose (48 mg) chosen for ZnCO₃ was below its effective level and that at higher concentrations/doses ZnCO₃ alone provides effective follicular antibacterial activity and underarm odor control.

In contrast, trials 2 and 1c demonstrate the potent follicular antibacterial efficacy of rinse off compositions containing particulate antimicrobial agent (zinc pyrithione (ZPT) alone) (Trial 2) or a particulate antimicrobial agent (ZPT) in combination with an additional antimicrobial agent (ZnCO₃) (Trial 1c). It can also be seen from the results of clinical trials 1 and 2 that the combination of ZPT and ZnCO₃ resulted in an enhanced level of follicular antibacterial control not anticipated from the results for the ZnCO₃ only (Trial 1) and ZPT only (Trial 2) clinical trials.

TABLE 1

| | | Dose | Baseline | | Change in |
|---|---|---|---|---|---|
| | ZPT/ZnCO3 | ZPT + | Detection | Day 10 | Baseline |
| | in the Body | ZnCO3 | Time | Detection | Detection |
| | Wash (%) | (mg) | (Hrs) | Time (Hrs) | Time (Hrs) |
| Trial | 0%/0% | 0 mg + 0 | 10.5 hrs | 10.6 hrs | 0.1 hrs |
| 1a | | mg | | | |
| Trial | 0%/1.6% | 0 mg + 48 | 11 hrs | 10.8 hrs | −0.2 hrs |
| 1b | | mg | | | |
| Trial | 1%/0% | 50 mg + 0 | 9.2 hrs | 19.9 hrs | 10.7 hrs |
| 2 | | mg | | | |
| Trial | 0.5%/1.6% | 15 mg + | 10.7 hrs | 28 hrs | 17.3 hrs |
| 1c | | 48 mg | | | |

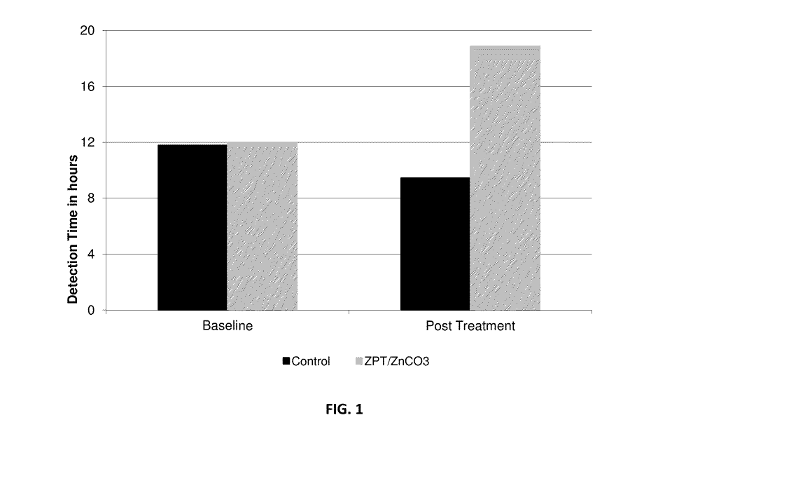

In addition, FIG. 1 shows the Hair Pluck (antibacterial) results for a third clinical trial (clinical 3) in which a 0.1% ZPT/0.5% ZnCO3 body wash composition was assessed relative to a body wash control composition that did not contain any particulate antimicrobial agent (ZPT or ZnCO₃). As can be seen, the ZPT/ZnCO₃ composition significantly (p<0.05) increased detection times versus the control composition indicating that the ZPT/ZnCO₃ composition had significantly stronger follicular antibacterial activity then the control.

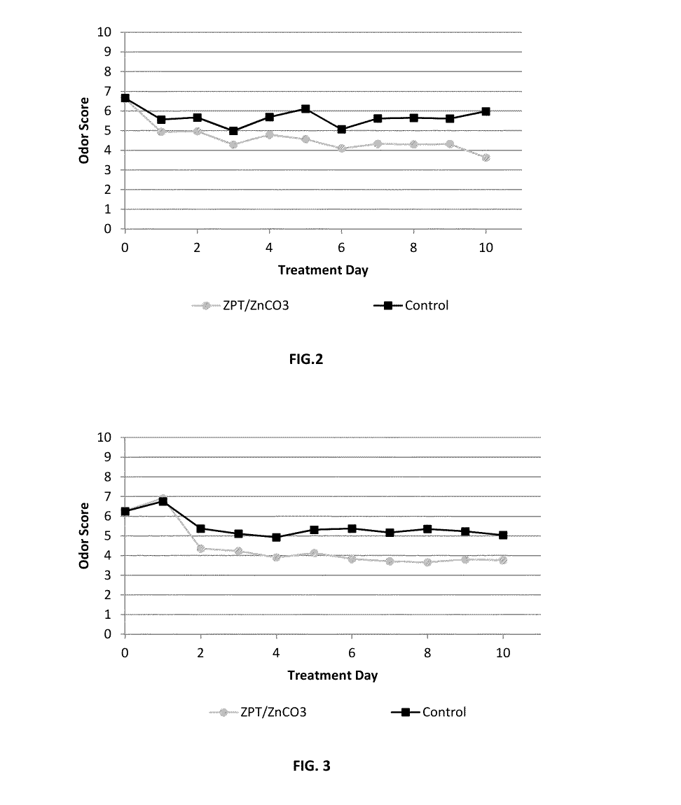

In addition to enhanced antimicrobial efficacy, clinical tests also show an improvement in odor. For example, for clinical trial 1 (n=46), FIGS. 2 and 3 demonstrate the 12 and 24 hour odor protection achieved through use of 0.5% ZPT/1.6% ZnCO₃ in a body wash chassis versus a control body wash chassis that does not contain any particulate antimicrobial agent. As can be seen, the ZPT/ZnCO₃ composition provided a statistically significant (p<0.05; repeated means) reduction in underarm odor at both 12 (FIG. 2) and 24 hours (FIG. 3) post use.

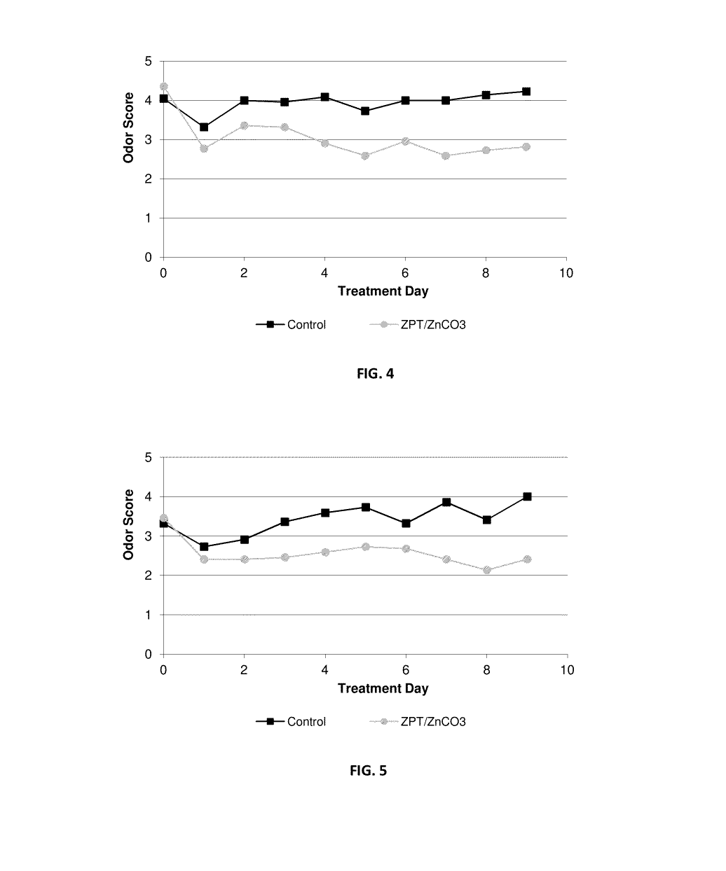

For clinical trial 3 (n=23), FIGS. 4 and 5 demonstrate the 12 and 24 hour odor protection achieved through use of 0.1% ZPT/0.5% ZnCO₃ in a body wash chassis versus a control body wash chassis that does not contain any particulate antimicrobial agent. As can be seen, the ZPT/ZnCO₃ composition provided a statistically significant (p<0.05; repeated means) reduction in underarm odor versus the control composition at both 12 (FIG. 4) and 24 hours (FIG. 5) post use.

As noted above, the particulate antimicrobial agents provided for herein substantially reduce the amount of microbial activity and thus the odor generated by the microbial activity and, in fact, provide longer lasting relief from the onset of malodor (e.g. longer than 24 hours). Often this activity is concentrated in particular locations on the body (e.g., underarm). Such particulate antimicrobial agents can be applied via a rinse-off personal care composition, and examples of such compositions are described herein.

### III. Rinse-Off Personal Care Compositions

Rinse-off personal care compositions will generally comprise a particulate antimicrobial agent. Rinse-off personal care compositions may come in many forms. For example, a personal care composition may be in a liquid form and could be a body wash, shampoo, conditioning shampoos, body washes, moisturizing body washes, shower gels, skin cleansers, cleansing milks, in shower body moisturizers, pet shampoos, shaving preparations, etc. Rinse-off personal care compositions may also be in a solid form, like in a bar soap. Bar soap can also be in many shapes and forms like a rectangle or in a powder or pellet form, for example.

Many personal care compositions can be water-based. It should be understood that an amount of water can be lost, i.e. evaporated, during a process of making a personal care composition, or subsequently, with water being absorbed by surrounding packaging (e.g. a cardboard carton), and the like. Thus, a personal care composition can also include materials that tend to bind the water such that the water can be maintained in the personal care composition at the desired levels. Examples of such materials can include carbohydrate structurants and humectants such as glycerin. However, it will be appreciated that a personal care composition can be anhydrous.

A variety of optional ingredients can also be added to a personal care composition. Such suitable ingredients can include, but are not limited to, structurants, humectants, fatty acids, inorganic salts, and other antimicrobial agents or actives.

A personal care composition can also optionally include hydrophilic structurants such as carbohydrate structurants and gums. Some suitable carbohydrate structurants include raw starch (corn, rice, potato, wheat, and the like) and pregelatinized starch. Some suitable gums include carregeenan and xanthan gum. A personal care composition may include from about 0.1% to about 30%, from about 2% to about 25%, or from about 4% to about 20%, by weight of the personal care composition, of a carbohydrate structurant.

A personal care composition can also optionally include one or more humectants. Examples of such humectants can include polyhydric alcohols. Further, humectants such as glycerin can be included the personal care composition as a result of production or as an additional ingredient. For example, glycerin can be a by-product after saponification of the personal care composition. Including additional humectant can result in a number of benefits such as improvement in hardness of the personal care composition, decreased water activity of the personal care composition, and reduction of a weight loss rate of the personal care composition over time due to water evaporation.

A personal care composition can optionally include inorganic salts. Inorganic salts can help to maintain a particular water content or level of the personal care composition and improve hardness of the personal care composition. The inorganic salts can also help to bind the water in the personal care composition to prevent water loss by evaporation or other means. A personal care composition can optionally include from about 0.01% to about 15%, from about 1% to about 12%, or from about 2.5% to about 10.5%, by weight of the personal care composition, of inorganic salt. Examples of suitable inorganic salts can include magnesium nitrate, trimagnesium phosphate, calcium chloride, sodium carbonate, sodium aluminum sulfate, disodium phosphate, sodium polymetaphosphate, sodium magnesium succinate, sodium tripolyphosphate, aluminum sulfate, aluminum chloride, aluminum chlorohydrate, aluminum-zirconium trichlorohydrate, aluminum-zirconium trichlorohydrate glycine complex, zinc sulfate, ammonium chloride, ammonium phosphate, calcium acetate, calcium nitrate, calcium phosphate, calcium sulfate, ferric sulfate, magnesium chloride, magnesium sulfate, and tetrasodium pyrophosphate.

A personal care composition can optionally further include one or more additional antibacterial agents that can serve to further enhance antimicrobial effectiveness of the personal care composition. A personal care composition can include, for example, from about 0.001% to about 2%, from about 0.01% to about 1.5%, or from about 0.1% to about 1%, by weight of the personal care composition, of additional antibacterial agent(s). Examples of suitable antibacterial agents can include carbanilides, triclocarban (also known as trichlorocarbanilide), triclosan, a halogenated diphenylether available as DP-300 from Ciba-Geigy, hexachlorophene, 3,4,5-tribromosalicylanilide, and salts of 2-pyridinethiol-1-oxide, salicylic acid, and other organic acids. Other suitable antibacterial agents are described in U.S. Pat. No. 6,488,943.

A. Liquid Personal Care Compositions

Exemplary liquid rinse-off personal care compositions can include an aqueous carrier, which can be present at a level of from about 5% to about 95%, or from about 60% to about 85%. The aqueous carrier may comprise water, or a miscible mixture of water and organic solvent. Non-aqueous carrier materials may also be employed.

Such rinse-off personal care compositions may include one or more detersive surfactants. The detersive surfactant component can be included to provide cleaning performance to the product. The detersive surfactant component in turn comprises anionic detersive surfactant, zwitterionic or amphoteric detersive surfactant, or a combination thereof. A representative, non-limiting, list of anionic surfactants includes anionic detersive surfactants for use in the compositions can include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In one example, the anionic surfactant can be sodium lauryl sulfate or sodium laureth sulfate. The concentration of the anionic surfactant component in the product can be sufficient to provide a desired cleaning and/or lather performance, and generally ranges from about 2% to about 50%.

Amphoteric detersive surfactants suitable for use in the rinse-off personal care compositions are well known in the art, and include those surfactants broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoactetate, disodium lauroamphoacetate disodium cocodiamphoacetate, and mixtures thereof. Amphoacetates and diamphoacetates can also be used.

Zwitterionic detersive surfactants suitable for use in the rinse-off personal care compositions are well known in the art, and include those surfactants broadly described as derivatives of aliphatic quaternary ammonium, phosphonium, and sulfonium compounds, in which aliphatic radicals can be straight or branched chains, and wherein an aliphatic substituent can contain from about 8 to about 18 carbon atoms such that one carbon atom can contain an anionic group, e.g., carboxy, sulfonate, sulfate, phosphate, or phosphonate. Other zwitterionic surfactants can include betaines, including cocoamidopropyl betaine.

The personal care composition can comprise one or more phases. Such personal care compositions can include a cleansing phase and/or a benefit phase (i.e., a single- or multi-phase composition). Each of a cleansing phrase or a benefit phase can include various components. The cleansing phase and the benefit phase can be blended, separate, or a combination thereof. The cleansing phase and the benefit phase can also be patterned (e.g. striped).

The cleansing phase of a personal care composition can include at least one surfactant. The cleansing phase may be an aqueous structured surfactant phase and be present at from about 5% to about 20%, by weight of the personal care composition. Such a structured surfactant phase may include sodium trideceth(n) sulfate, hereinafter STnS, wherein n can define average moles of ethoxylation. n can range, for example, from about 0 to about 3; from about 0.5 to about 2.7, from about 1.1 to about 2.5, from about 1.8 to about 2.2, or n can be about 2. When n can be less than 3, STnS can provide improved stability, improved compatibility of benefit agents within the personal care compositions, and increased mildness of the personal care compositions, such described benefits of STnS are disclosed in U.S. patent application Ser. No. 13/157,665.

The cleansing phase can also comprise at least one of an amphoteric surfactant and a zwitterionic surfactant. Suitable amphoteric or zwitterionic surfactants (in addition to those cited herein) can include, for example, those described in U.S. Pat. No. 5,104,646 and U.S. Pat. No. 5,106,609.

A cleansing phase can comprise a structuring system. A structuring system can comprise, optionally, a non-ionic emulsifier, optionally, from about 0.05% to about 5%, by weight of the personal care composition, of an associative polymer; and an electrolyte.

The personal care composition can be optionally free of sodium lauryl sulfate, hereinafter SLS, and can comprise at least a 70% lamellar structure. However, the cleansing phase could comprise at least one surfactant, wherein the at least one surfactant includes SLS. Suitable examples of SLS are described in U.S. patent application Ser. No. 12/817,786.

As noted herein, rinse-off personal care compositions can also include a benefit phase. The benefit phase can be hydrophobic and/or anhydrous. The benefit phase can also be substantially free of surfactant. A benefit phase can also include a benefit agent. In particular, a benefit phase can comprise from about 0.1% to about 50%, by weight of the personal care composition, of the benefit agent. The benefit phase may comprise less benefit agent, for example, from about 0.5% to about 20%, by weight of the personal care composition, of the benefit agent. Examples of suitable benefit agents can include petrolatum, glyceryl monooleate, mineral oil, natural oils, and mixtures thereof. Additional examples of benefit agents can include water insoluble or hydrophobic benefit agents. Other suitable benefit agents are described in U.S. patent application Ser. No. 13/157,665.

Non-limiting examples of glycerides suitable for use as hydrophobic skin benefit agents herein can include castor oil, safflower oil, corn oil, walnut oil, peanut oil, olive oil, cod liver oil, almond oil, avocado oil, palm oil, sesame oil, vegetable oils, sunflower seed oil, soybean oil, vegetable oil derivatives, coconut oil and derivatized coconut oil, cottonseed oil and derivatized cottonseed oil, jojoba oil, cocoa butter, and combinations thereof.

Non-limiting examples of alkyl esters suitable for use as hydrophobic skin benefit agents herein can include isopropyl esters of fatty acids and long chain esters of long chain (i.e. C10-C24) fatty acids, e.g., cetyl ricinoleate, non-limiting examples of which can include isopropyl palmitate, isopropyl myristate, cetyl riconoleate, and stearyl riconoleate. Other example can include hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of polyglycerin fatty acid esters suitable for use as hydrophobic skin benefit agents herein can include decaglyceryl distearate, decaglyceryl diisostearate, decaglyceryl monomyriate, decaglyceryl monolaurate, hexaglyceryl monooleate, and combinations thereof.

The rinse-off personal care composition may be applied by a variety of means, including by rubbing, wiping or dabbing with hands or fingers, or by means of an implement and/or delivery enhancement device. Non-limiting examples of implements include a sponge or sponge-tipped applicator, a mesh shower puff, a swab, a brush, a wipe (e.g., wash cloth), a loofah, and combinations thereof. Non-limiting examples of delivery enhancement devices include mechanical, electrical, ultrasonic and/or other energy devices. Employment of an implement or device may help delivery of the particulate antimicrobial agent to target regions, such as, for example, hair follicles and undulations that can exist in the underarm. The rinse-off care product may be sold together with such an implement or device. Alternatively, an implement or device can be sold separately but contain indicium to indicate usage with a rinse-off care product. Implements and delivery devices can employ replaceable portions (e.g., the skin interaction portions), which can be sold separately or sold together with the rinse-off care product in a kit.

B. Solid Personal Care Compositions

As noted herein, personal care compositions can take on numerous forms. One suitable form is that of a solid personal care composition. Solid compositions can take many forms like powder, pellets, bars, etc. These forms will generally be described herein as bar soap, but it should be understood that the solid composition could be in another form or shape. One example of a bar soap personal care composition can include from about 0.1% to about 35%, by weight of the personal care composition, of water, from about 45% to about 99%, by weight of the personal care composition, of soap, and from about 0.01% to about 5%, by weight of the personal care composition, of a particulate antimicrobial agent. Another suitable antimicrobial bar soap can include , for example, from about 0.1% to about 30%, by weight of the personal care composition, of water, from about 40% to about 99%, by weight of the personal care composition, of soap, and from about 0.25% to about 3%, by weight of the personal care composition, of a particulate antimicrobial agent.

Bar soap compositions can be referred to as conventional solid (i.e. non-flowing) bar soap compositions. Some bar soap composition comprise convention soap, while others contain synthetic surfactants, and still others contain a mix of soap and synthetic surfactant. Bar compositions may include, for example, from about 0% to about 45% of a synthetic anionic surfactant. An example of a suitable conventional soap can include milled toilet bars that are unbuilt (i.e. include about 5% or less of a water-soluble surfactancy builder).

A personal care bar composition can include, for example from about 45% to about 99% or from about 50% to about 75%, by weight of the personal care composition, of soap. Such soaps can include a typical soap, i.e., an alkali metal or alkanol ammonium salt of an alkane- or alkene monocarboxylic acid. Sodium, magnesium, potassium, calcium, mono-, di- and tri-ethanol ammonium cations, or combinations thereof, can be suitable for a personal care composition. The soap included in a personal care composition can include sodium soaps or a combination of sodium soaps with from about 1% to about 25% ammonium, potassium, magnesium, calcium, or a mixture of these soaps. Additionally, the soap can be well-known alkali metal salts of alkanoic or alkenoic acids having from about 12 to about 22 carbon atoms or from about 12 to about 18 carbon atoms. Another suitable soap can be alkali metal carboxylates of alkyl or alkene hydrocarbons having from about 12 to about 22 carbon atoms. Additional suitable soap compositions are described in U.S. patent application Ser. No. 13/036,889.

A personal care composition can also include soaps having a fatty acid. For example, one bar soap composition could use from about 40% to about 95% of soluble alkali metal soap of C₈-C₂₄ or C₁₀-C₂₀ fatty acids. The fatty acid may, for example, have a distribution of coconut oil that can provide a lower end of a broad molecular weight range or a fatty acid distribution of peanut or rapeseed oil, or their hydrogenated derivatives, which can provide an upper end of the broad molecular weight range. Other such compositions can include a fatty acid distribution of tallow and/or vegetable oil. The tallow can include fatty acid mixtures that can typically have an approximate carbon chain length distribution of 2.5% C₁₄, 29% C₁₆, 23% C₁₈, 2% palmitoleic, 41.5% oleic, and 3% linoleic. The tallow can also include other mixtures with a similar distribution, such as fatty acids derived from various animal tallows and/or lard. In one example, the tallow can also be hardened (i.e., hydrogenated) such that some or all unsaturated fatty acid moieties can be converted to saturated fatty acid moieties.

Suitable examples of vegetable oil include palm oil, coconut oil, palm kernel oil, palm oil stearine, soybean oil, and hydrogenated rice bran oil, or mixtures thereof, since such oils can be among more readily available fats. One example of a suitable coconut oil can include a proportion of fatty acids having at least 12 carbon atoms of about 85%. Such a proportion can be greater when mixtures of coconut oil and fats such as tallow, palm oil, or non-tropical nut oils or fats can be used where principle chain lengths can be C₁₆ and higher. The soap included in a personal care composition can be, for example, a sodium soap having a mixture of about 67-68% tallow, about 16-17% coconut oil, about 2% glycerin, and about 14% water.

Soap included in a personal care composition can also be unsaturated in accordance with commercially acceptable standards. For example, a soap included in a personal care composition could include unsaturation in a range of from about 37% to about 45% of saponified material.

Soaps included in a personal care composition can be made, for example, by a classic kettle boiling process or modern continuous soap manufacturing processes wherein natural fats and oils such as tallow or coconut oil or their equivalents can be saponified with an alkali metal hydroxide using procedures well known to those skilled in the art. Soap can also be made by neutralizing fatty acids such as lauric (C₁₂), myristic (C₁₄), palmitic (C₁₆), or stearic (C₁₈) acids, with an alkali metal hydroxide or carbonate.

Soap included in a personal care composition could also be made by a continuous soap manufacturing process. The soap could be processed into soap noodles via a vacuum flash drying process. One example of a suitable soap noodle comprises about 67.2% tallow soap, about 16.8% coconut soap, about 2% glycerin, and about 14% water, by weight of the soap noodle. The soap noodles can then be utilized in a milling process to finalize a personal care composition.

### IV. Antimicrobial Agents

As noted herein, rinse-off personal care compositions can further include antimicrobial agents. When in a phased personal care composition, such antimicrobial agents can be found in the cleansing phase and/or benefit phase of the personal care composition. Exemplary rinse-off personal care compositions can employ a particulate antimicrobial agent. The antimicrobial agent and carrier material (and/or other solvent-acting material ingredients) can be chosen such that the antimicrobial agent remains as a solid particulate within the final formulation and upon application to the skin; that is, the antimicrobial agent is not completely solubilized prior to use. Remaining in particulate form within the final formulation and upon application to the skin enables at least a portion of the antimicrobial agent to deposit into skin crevices and/or hair follicles, and to survive rinsing. Microscopy can enable one to determine the presence of discrete antimicrobial agent particles within the final formulation. The antimicrobial agent particles can range from completely insoluble in the personal care composition; to substantially insoluble in the composition; to less than 5% soluble; to less than 1% soluble, by weight of the antimicrobial agent particles.

Antimicrobial agent particles generally range in particle size from about 0.1 μm to about 100 μm and can be even smaller like, from about 0.2 μm to about 50 μm; from about 0.5 μm to about 20 μm; or from about 1 μm to about 10 μm. It should be appreciated that not all of the plurality of antimicrobial agent particles within a given product necessarily falls within the above range and that the particle distribution may be normal or not. It is believed that antimicrobial particles in the 0.1 μm to 10 μm size range can deposit into hair follicles, while larger particles (e.g., 10-100 μm or even larger) may deposit into skin folds, wrinkles, crevices or other surface irregularities that can be present. Particles that are larger than 100 μm may be perceptible during application and impart a gritty feel. Some consumers may desire the tactile feedback with larger particles, believing that the grittiness is helping to remove unwanted material from the skin; thus at least some particles larger than 100 μm can be used in a personal care composition if desired. If it is desired to reduce or eliminate the gritty feel of larger particles, antimicrobial agent particles larger than 100 μm are minimized or absent altogether.

Particle size can be measured using light scattering methods. Specifically, particle size can be determined with a Horiba LA-950 Laser Diffraction Particle Size Analyzer. This instrument uses the principal of low-angle Fraunhofer Diffraction and Light Scattering from the particles as the means for particle size determination. A low-power, visible laser produces a collimated, monochromatic beam of light that illuminates the sample. The incident light is diffracted by the particles illuminated to give a nearly stationary diffraction pattern regardless of particle movement. The incident light is subsequently collected by a receiver lens and imaged onto a multi-element solid-state photo detector. As particles enter and leave the illuminated area, the diffraction pattern evolves, always reflecting the instantaneous size distribution in this area. As the laser illuminates the sample, the particles scatter light in all directions around the sample. During the experiment, a lamp is also used to illuminate the sample, scattering off the particles in all directions around the sample. The scattered light from the laser and the lamp is collected onto light sensors that are positioned at multi-angles relative to the sample. The resulting scattering pattern is a function of the particle size in the sample. The detectors are interfaced directly into a desk-top computer which analyzes the diffraction and scattering patterns. The computer uses a method of non-linear least squares analysis to find the size distribution that gives the closest fit to the diffraction and scattering pattern. The instrument then provides the particle size analysis results, which includes a particle size distribution graph and various quantitative parameters that describe the particle size distribution. This method is applicable to a wide range of particles suspended in water and/or formulation slurries.

Staphylococcus epidermidis and corynebacterium mucifaciens are two of the key odor-causing bacteria associated with the human underarm. Accordingly, when rinse-off personal care compositions are designed for managing underarm malodor, the antimicrobial agent can have a Minimal Inhibitory Concentration (“MIC”) of less than or equal to 2,500 μg/ml, 1,000 μg/ml, 500 μg/ml, or 100 μg/ml against at least one strain of at least one of these bacteria. MIC values can be obtained using a traditional broth dilution microbiological technique, such as that described in the following journal article: Andrews, J. M., “Determination of minimum inhibitory concentrations”, Journal of Antimicrobial Chemotherapy 48 (supl. 1): 5-16, 2001.

Suitable antimicrobial agents can include, but are not limited to, metals (e.g., Zn, Cu, Al, Ti, Sn, Bi, and Ag), metal salts (e.g., zinc carbonate, copper sulfate, and zinc gluconate), metal pyrithione salts (e.g., ZPT and CuPT), zeolites, metal zeolites, quaternary ammonium (quat) compounds (e.g., cetyl pyridinium chloride, and benzylalkonium chloride), quat bound clays, metal bound clays, and PolyAspirin (e.g., as described in PCT publication no. WO 2008/034019). An antimicrobial agent can be employed in the rinse-off personal care compositions at levels of from about 0.01% to about 10%; other levels may however also be possible. Due to differences in the antibacterial potency of the various particulate antibacterial agents formulation levels may need to be adjusted accordingly. For example, compositions containing zinc carbonate alone may need to be formulated at the upper range of the listed concentration (2-10%) to achieve optimum odor and follicular bacterial control.

A rinse-off personal care composition can contain a zinc-containing antimicrobial agent. Such agents can include, for example, a zinc salt of 1-hydroxy-2-pyridinethione (known as “zinc pyrithione” or “ZPT”), for example, a mercaptopyridine-N-oxide zinc salt. The ZPT can be made by reacting 1-hydroxy-2-pyridinethione (i.e., pyrithione acid) or a soluble salt thereof with a zinc salt (e.g. zinc sulfate) to form a zinc pyrithione precipitate as illustrated in U.S. Pat. No. 2,809,971 and the zinc pyrithione can be formed or processed into platelet ZPT using, for example, sonic energy as illustrated in U.S. Pat. No. 6,682,724.

Zinc pyrithione can take the form of particulates, platelets, or a combination thereof. Particulate ZPT can, for example, have an average particle size from about 0.1 μm to about 20 μm or from about 0.2 μm to about 10 μm.

Other suitable examples of zinc salts useful herein can include the following: zinc aluminate, zinc carbonate, zinc oxide, zinc phosphates, zinc selenide, zinc sulfide, zinc silicates, zinc silicofluoride, zinc borate, zinc hydroxide, zinc hydroxy sulfate, and combinations thereof.

Other non-limiting zinc containing materials can include zinc-containing layer materials (“ZLM's”). ZLM's can typically be those materials with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). ZLM's may have zinc incorporated in the layers and/or be components of the gallery ions. Other suitable ZLMs are described in U.S. Patent Application Publication No. 2008/0138441.

Many ZLM's occur naturally as minerals. Common examples include hydrozincite (zinc carbonate hydroxide), basic zinc carbonate, aurichalcite (zinc copper carbonate hydroxide), rosasite (copper zinc carbonate hydroxide) and many related minerals that are zinc-containing. Natural ZLM's can also occur wherein anionic layer species such as clay-type minerals (e.g., phyllosilicates) contain ion-exchanged zinc gallery ions. All of these natural materials can also be obtained synthetically or formed in situ in a product or during a production process.

Another common class of ZLM's, which are often, but not always, synthetic, is layered doubly hydroxides, which are generally represented by the formula [M²⁺₁₋ₓM³⁺ₓ(OH)₂]^{x+} A^{m-}_{x/m}.nH₂O and some or all of the divalent ions (M²⁺) would be represented as zinc ions (Crepaldi, E L, Pava, P C, Tronto, J, Valim, J B J. Colloid Interfac. Sci. 2002, 248, 429-42).

Yet another class of ZLM's can be prepared called hydroxy double salts (Morioka, H., Tagaya, H., Karasu, M, Kadokawa, J, Chiba, K Inorg. Chem. 1999, 38, 4211-6). Hydroxy double salts can be represented by the general formula [M²⁺₁₋ₓM²⁺₁₊ₓ(OH)_{3(1-y)}]⁺ Aⁿ⁻_{(1=3y)/n}.nH₂O where the two metal ion may be different; if they are the same and represented by zinc, the formula simplifies to [Zn₁₊ₓ(OH)₂]^{2x+}2× A⁻.nH₂O. This latter formula represents (where x=0.4) common materials such as zinc hydroxychloride and zinc hydroxynitrate. These are related to hydrozincite as well wherein a divalent anion replace the monovalent anion. These materials can also be formed in situ in a product or in or during a production process.

These classes of ZLM' s represent relatively common examples of the general category and are not intended to be limiting as to the broader scope of materials which fit this definition.

Commercially available sources of basic zinc carbonate include Zinc Carbonate Basic (Cater Chemicals: Bensenville, Ill., USA), Zinc Carbonate (Shepherd Chemicals: Norwood, Ohio, USA), Zinc Carbonate (CPS Union Corp.: New York, N.Y., USA), Zinc Carbonate (Elementis Pigments: Durham, UK), and Zinc Carbonate AC (Bruggemann Chemical: Newtown Square, Pa., USA).

Basic zinc carbonate, which also may be referred to commercially as “Zinc Carbonate” or “Zinc Carbonate Basic” or “Zinc Hydroxy Carbonate”, is a synthetic version consisting of materials similar to naturally occurring hydrozincite. The idealized stoichiometry is represented by Zn₅(OH)₆(CO₃)₂ but the actual stoichiometric ratios can vary slightly and other impurities may be incorporated in the crystal lattice.

A personal care composition can contain a combination of antibacterial agents. For example, a combination of ZPT and a ZLM (such as, for example, basic zinc carbonate) can be used. One exemplary combination includes from about 0.025% to about 0.5% particulate zinc pyrithione and from about 0.1% to about 2.0% of zinc carbonate.

Additionally, a personal care composition can include a combination of ZPT and other zinc salts including, for example, the following: zinc aluminate, zinc carbonate, zinc oxide, zinc phosphates, zinc selenide, zinc sulfide, zinc silicates, zinc silicofluoride, zinc borate, zinc hydroxide, zinc hydroxy sulfate, and combinations thereof.

The combination of zinc-containing materials can provide enhanced antimicrobial benefits and synergistic effects (e.g., improved antibacterial efficacy). In fact, using particulate antimicrobial agents having a combination of zinc-containing materials (e.g, zinc pyrithione and zinc carbonate) in a rinse-off personal care composition can further control odor reduction. This can be seen from the results of clinical trials 1 and 2 where the combination of ZPT and ZnCO₃ resulted in an enhanced level of follicular antibacterial control not anticipated from the results for the ZnCO₃ only (Trial 1) and ZPT only (Trial 2) clinical trials. This synergistic effect can result, for example, in controlling odor for at least 12 hours after application of the rinse-off personal care composition, or even for at least 24 hours after application of the rinse-off personal care composition, or even for at least 48 hours after application of the rinse-off personal care composition.

A personal care composition can optionally further include one or more additional antibacterial agents that can serve to further enhance antimicrobial effectiveness of the personal care composition. When present, a personal care composition can include from about 0.001% to about 2%, from about 0.01% to about 1.5%, or from about 0.1% to about 1%, by weight of the personal care composition, of an additional antibacterial agent. Examples of antibacterial agents can include carbanilides, for example, triclocarban (also known as trichlorocarbanilide), triclosan, a halogenated diphenylether available as DP-300 from Ciba-Geigy, hexachlorophene, 3,4,5-tribromosalicylanilide, and salts of 2-pyridinethiol-1-oxide, salicylic acid, and other organic acids.

Other suitable antimicrobial agetns can include quaternary ammonium compounds (such as cetylpyridinium chloride), coal tar, sulfur, whitfield's ointment, castellani's paint, aluminum chloride, gentian violet, octopirox (piroctone olamine), ciclopirox olamine, undecylenic acid and it's metal salts, potassium permanganate, selenium sulfide, sodium thiosulfate, propylene glycol, oil of bitter orange, urea preparations, griseofulvin, 8-Hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone and azoles, and combinations thereof. Suitable antibacterial agents are described in U.S. Pat. No. 6,488,943 and U.S. Patent Application Publication No. 2008/0138441.

### V. Methods

As discussed above, improved methods for reducing malodor are included herein. For brevity, only a couple of exemplary methods and compositions are described in this section, but any of the properties or characteristics noted above could be used in the methods.

One exemplary method comprises depositing at least a portion of a particulate antimicrobial agent into a hair follicle, wherein the particulate antimicrobial agent has an average particle size less than about 50 μm and the particulate antimicrobial agent is applied to the hair follicle via a rinse-off personal care composition. The method may further comprise depositing at least a portion of the particulate antimicrobial material into a skin crevice and/or controlling odor for at least 12 hours after application of the rinse-off composition. The average particle size may be even smaller, for example, less than about 20 μm or less than about 10 μm.

The particulate antimicrobial agent may be less than about 5% soluble in the rinse-off composition. The particulate antimicrobial agent may comprise a zinc containing material. The zinc containing material may comprise zinc pyrithione. The zinc pyrithione may be present from about 0.01% to about 4%, by weight of the rinse-off composition. The zinc containing material may further comprise a zinc salt. The zinc salt may comprise zinc carbonate. The zinc carbonate may be present from about 0.01% to about 5%, by weight of the rinse-off composition.

The rinse-off composition may comprise a detersive surfactant. The personal care composition may, for example, be in the form of a body wash or bar soap. The rinse-off composition may be substantially free of an antiperspirant.

Another exemplary method comprises applying a rinse-off personal care composition to at least a portion of hair follicles on skin, wherein the rinse-off personal cleansing composition comprises from about 0.025% to about 0.5% particulate zinc pyrithione and from about 0.5% to about 1.5% of zinc carbonate and wherein the zinc pyrithione has an average particle size of less than 10 μm and zinc carbonate has an average particle size less than about 50 μm. The method may further comprise depositing at least a portion of the particulate antimicrobial agent into hair follicles. The average particle size of the zinc pyrithione may be even smaller at less than about 5 μm. Less than about 5% of the zinc pyrithione and the zinc carbonate may, for example, be soluble in the rinse-off composition. The rinse-off composition may be in the form of a body wash or a bar soap.

### VI. Procedure—Hair Pluck and Self Assessed Body Odor Clinical Methods and Results

Individuals were subject to a 3-day washout period where no body wash, antiperspirant, or deodorant was used and only soap and shampoo usage was permitted. Following the 3-day washout period, a treatment period followed. For 9-10 consecutive days, the individual's underarm was limited to one treatment per day with the given tested trial product, and the individual was not permitted to use any other soap, body wash, antiperspirant or deodorant product. The dose of the tested trial product (e.g., rinse off personal care composition) was limited to between 1-5 g of test product per underarm. Following treatment with the rinse off personal care composition and to simulate real world product usage, the subject's underarm was thoroughly rinsed off with tap water until all visible lather was removed. Both the antibacterial activity (Hair Plucking) and anti-odor (Self Assessments) efficacy of the compositions were determined simultaneously in the same studies.

For Assessment of Antibacterial Activity: Replicate hairs were plucked and removed from the underarm at baseline (end of the washout period) and about 5 hours after the final (9^{th} or 10^{th}) treatment and placed individually into separate Soleris vials. The vials were incubated at 37° C. in a Soleris photometer and continuously monitored for 48 hours. Follicular associated bacteria present on the hair metabolized carbohydrates in the vials generating acid byproducts, decreasing the culture pH, and causing a color change in a pH indicator present in the vial. The time (hours) to color change, known as the detection time, is inversely proportional to the number and/or metabolic health of follicular bacteria carried over on the hair. That is, the longer the detection time, the fewer in number or less “healthy” the follicular bacteria. Longer detection times are indicative of a strong antibacterial effect within the hair follicle while shorter times suggest a weak or non-existent antibacterial effect. Strong follicular antibacterial control is associated with strong underarm malodor control while weak follicular antibacterial control is associated with weak to nonexistent underarm malodor control.

For Assessment of Anti-Odor Efficacy: Subjects self assessed their underarm odor at baseline and twice/day (am & pm) throughout the clinical trial. For their individualized odor assessment, each subject removed their shirt, turned their head towards the underarm and sniffed. An odor score was assigned for each underarm using a 0-10 rating scale where 0 indicated no detectable odor and 10 represented the highest level of odor this subject had personally experienced on themselves. Underarm odor scores were designated as “12 hour” and “24 hour”; where “12 hour” scores represent odor measures taken in the evening, or about 12 hours after product use and “24 hour” scores represent odor measures taking the following morning, or about 24 hours after product use.

### EXAMPLES

### Rinse off Personal Care Compositions

Table 2 shows some non-limiting examples of inventive rinse off personal care formulations.

TABLE 2

| | Body Wash | Body Wash | Bar Soap |
|---|---|---|---|
| | 0.25% ZPT + | 0.1% ZPT + | 0.25% ZPT + |
| Ingredient | 1.0% ZnCO3 | 0.5% ZnCO₃ | 1.0% ZnCO₃ |
| Water | 60.72 | 61.6488 | 1.95 |
| Sodium Laureth-3- | 18.76 | | |
| Sulfate | | | |
| Sodium Lauryl Sulfate | 11.65 | | |
| Cocoamidopropyl | 4.47 | | |
| Betaine | | | |
| Sodium Chloride | 1.40 | 1.5700 | |
| Zinc Carbonate + | 1.06 | 0.5000 | 1.00 |
| Mg Carbonate Hydroxide | | | |
| Hydrochloric | 0.85 | 0.3750 | |
| Acid, >25% and fuming | | | |
| Zinc Omadine 48% | 0.52 | 0.2045 | 0.51 |
| FPS Cosmetic Grade | | | |
| Thixcin R | 0.37 | | |
| NaOH | 0.17 | | |
| Kathon CG | 0.03 | 0.0330 | |
| SLE1S 26% Active | | 28.8000 | |
| High pH FFR | | | |
| Sodium Benzoate, nf | | 0.2500 | |
| Cetyl Alcohol | | 0.0418 | |
| Stearyl Alcohol | | 0.0752 | |
| Glycol Distearate | | 1.4950 | |
| (EGDS) | | | |
| Amphosol HCA-HP | | 5.0000 | |
| Caustic Soda 50% | | 0.0067 | |
| (Rayon Grade) | | | |
| Generic Dried | | | 96.54 |
| Noodle/Soap | | | |
| Total % | 100.00 | 100.00 | 100.00 |

Compositions were produced using either standard lab or pilot plant scale formulation processes. Specifically, for the body wash compositions, a pre-mix was prepared by combining distilled water with the surfactants, thickeners and preservative ingredients. The resulting solution was mixed at room temperature until uniform. ZPT, ZnCO₃, and sodium chloride were then added and mixed into the solution until homogeneously distributed throughout. The resulting slurry was then adjusted to the final desired pH (˜7.0) as needed. For the Bar Soap compositions, standard dried soap flakes, ZPT, and ZnCO₃ particles were combined into an amalgamator and transferred to a 3-roll mill for micro mixing (3 passes). Upon completion of milling, the flakes were transferred to a plodder (plodder jacket, mill roll, feed and transfer temperatures ˜110° F.; final temperature ˜80° F.) and extruded into plugs for stamping into appropriately sized soap bars.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The products and methods/processes of the present disclosure can comprise, consist of, and consist essentially of the essential elements and limitations of the invention described herein, as well as any of the additional or optional ingredients, components, steps, or limitations described herein.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of reducing odor, comprising:
depositing at least a portion of a particulate antimicrobial agent into a hair follicle, wherein the particulate antimicrobial agent has an average particle size less than about 100 μm and the particulate antimicrobial agent is applied to the hair follicle via a rinse-off personal care composition.

2. The method of claim 1, wherein the average particle size is less than about 50 μm.

3. The method of claim 1, wherein the average particle size is less than about 20 μm.

4. The method of claim 1, wherein the particulate antimicrobial agent comprises at least one zinc-containing material.

5. The method of claim 4, wherein the at least one zinc-containing material comprises zinc pyrithione.

6. The method of claim 5, wherein the rinse-off personal care composition comprises from about 0.01% to about 4%, by weight of the rinse-off personal care composition, of zinc pyrithione.

7. The method of claim 4, wherein the at least one zinc-containing material further comprises a zinc salt.

8. The method of claim 7, wherein the zinc salt comprises zinc carbonate.

9. The method of claim 8, wherein the rinse-off personal care composition comprises from about 0.1% to about 10% of zinc carbonate.

10. The method of claim 1, wherein the rinse-off personal care composition comprises at least one detersive surfactant.

11. The method of claim 1, wherein less than about 5% of the particulate antimicrobial agent is soluble in the rinse-off personal care composition.

12. The method of claim 1, further comprising depositing at least a portion of the particulate antimicrobial agent into a skin crevice.

13. The method of claim 1, wherein the rinse-off composition comprises a body wash or a bar soap.

14. The method of claim 1, wherein the rinse-off personal care composition is substantially free of antiperspirant.

15. The method of claim 1, further comprising controlling odor for at least 12 hours after application of the rinse-off personal care composition.

16. A method of reducing odor, comprising:
applying a rinse-off personal care composition to at least a portion of hair follicles on skin, wherein the rinse-off personal cleansing composition comprises from about 0.025% to about 1.0% particulate zinc pyrithione and from about 0.2% to about 2.0% of zinc carbonate and wherein the zinc pyrithione has an average particle size of less than about 10 um and zinc carbonate has an average particle size less than about 50 μm.

17. The method of claim 16, wherein the average particle size of the zinc pyrithione is less than about 5 μm.

18. The method of claim 17, wherein further comprising depositing at least a portion of the particulate antimicrobial agent into hair follicles

19. The method of claim 18, wherein less than about 5% of the zinc pyrithione and zinc carbonate is soluble in the rinse-off personal care composition.

20. The method of claim 19, wherein the rinse-off composition comprises a body wash or a bar soap.
